(19) United States
(12) Patent Application Publication
(10) Pub. No.: US 2025/0127427 A1
(43) Pub. Date: Apr. 24, 2025

(54) MOTION STATE MONITORING SYSTEM, MOTION STATE MONITORING METHOD, AND NON-TRANSITORY COMPUTER READABLE MEDIUM STORING MOTION STATE MONITORING PROGRAM

(71) Applicant: TOYOTA JIDOSHA KABUSHIKI KAISHA, Toyota-shi (JP)

(72) Inventors: Makoto KOBAYASHI, Nisshin-shi (JP); Yoshiaki KATO, Miyoshi-shi (JP); Taiga MATSUMOTO, Nagoya-shi (JP)

(73) Assignee: TOYOTA JIDOSHA KABUSHIKI KAISHA, Toyota-shi (JP)

(21) Appl. No.: 18/822,696

(22) Filed: Sep. 3, 2024

(30) Foreign Application Priority Data
Oct. 24, 2023 (JP) 2023-182853

(51) Int. Cl. A61B 5/11; A61B 5/00

(52) U.S. Cl. CPC A61B 5/1128; A61B 5/0077; A61B 5/743; A61B 5/744; A61B 2576/00

(57) ABSTRACT

A motion state monitoring system according to the present disclosure is configured to monitor a motion state at a target part of the body of a subject, and include: an arithmetic processing unit configured to generate a calculation result representing the motion state based on a detection result of a sensor configured to detect the motion state; an image processing unit configured to perform image processing on at least one of a photographed image obtained by photographing the motion state and a drawn image obtained by drawing the motion state; and a display unit configured to display the image-processed image and the calculation result in synchronization with each other.

### CROSS REFERENCE TO RELATED APPLICATIONS

This application is based upon and claims the benefit of priority from Japanese patent application No. 2023-182853, filed on Oct. 24, 2023, the disclosure of which is incorporated herein in its entirety by reference.

### BACKGROUND

The present disclosure relates to a motion state monitoring system, a motion state monitoring method, and a motion state monitoring program.

Japanese Unexamined Patent Application Publication No. 2022-034450 discloses a motion state monitoring system that monitors a motion state at a target part of a subject's body. A display unit in the motion state monitoring system disclosed in Japanese Unexamined Patent Application Publication No. 2022-034450 displays an icon indicating the position of a sensor that detects the motion state and a line graph in which the motion state detected by the sensor is displayed as an angle measured from a predetermined position.

### SUMMARY

It is difficult to comprehend the motion state at the target part of the subject's body based solely on the angle shown in the line graph when data thereon is reviewed. It has been desired to facilitate the comprehension of the motion state at the target part of the subject's body.

The present disclosure has been made in view of the above-described circumstances, and an object thereof is to provide a motion state monitoring system, a motion state monitoring method, and a motion state monitoring program capable of facilitating the comprehension of a motion state at a target part of a subject's body when data thereon is reviewed.

A motion state monitoring system according to an aspect of the present disclosure is a motion state monitoring system configured to monitor a motion state at a target part of a subject's body, including: an arithmetic processing unit configured to generate a calculation result representing the motion state based on a detection result of a sensor configured to detect the motion state; an image processing unit configured to perform image processing on at least one of a photographed image obtained by photographing the motion state and a drawn image obtained by drawing the motion state; and a display unit configured to display the image-processed image and the calculation result in synchronization with each other. The arithmetic processing unit may perform arithmetic processing by using a trained model generated through machine learning using a past detection result of the sensor.

In the above-described motion state monitoring system, the image processing unit may generate the drawn image of a 3D model obtained by drawing the motion state or the drawn image of an avatar obtained by drawing the motion state, and the display unit may display the generated drawn image of the 3D model.

In the above-described motion state monitoring system, the image processing unit may generate a locus of the target part in the drawn image of the 3D model or the avatar, and the display unit may display the drawn image including the generated locus.

In the above-described motion state monitoring system, the image processing unit may generate a locus of the target part in the photographed image, and the display unit may display the photographed image including the generated locus.

In the above-described motion state monitoring system, the image processing unit may generate a locus of the target part in the drawn image of the 3D model or the avatar, obtained by drawing the motion state shown in the photographed image, and the display unit may display the drawn image including the generated locus.

In the above-described motion state monitoring system, the image processing unit may generate a locus of the target part in the drawn image of the 3D model or the avatar, obtained by drawing the motion state, the motion state being generated in advance and used as a reference, and the display unit may display the drawn image including the generated locus.

In the above-described motion state monitoring system, the image processing unit may: generate a first locus of the target part in the drawn image of the 3D model or the avatar, obtained by drawing the motion state shown in the photographed image; generate a second locus of the target part in the drawn image of the 3D model or the avatar, obtained by drawing the motion state, the motion state being generated in advance as the reference; and generate a Lissajous figure in which the first and second loci are combined with each other, and the display unit may display the generated Lissajous figure.

In the above-described motion state monitoring system, the display unit may display the drawn image of the 3D model or the avatar in an enlarged or reduced manner.

In the above-described motion state monitoring system, the image processing unit may generate a figure indicating a movable area of the target part in the drawn image of the 3D model or the avatar, and the display unit may display the generated figure indicating the movable area.

In the motion state monitoring system, the display unit may further display at least one of vital data and a walking speed.

In the motion state monitoring system, the display unit may display a plurality of images obtained at different times.

A motion state monitoring method according to an aspect of the present disclosure is a motion state monitoring method for monitoring a motion state at a target part of a subject's body by using a motion state monitoring system, the motion state monitoring system including: an arithmetic processing unit configured to generate a calculation result representing the motion state based on a detection result of a sensor configured to detect the motion state; an image processing unit configured to perform image processing on at least one of a photographed image obtained by photographing the motion state and a drawn image obtained by drawing the motion state; and a display unit configured to display the image-processed image and the calculation result in synchronization with each other, and the motion state monitoring method including: generating the calculation result representing the motion state based on the detection result; performing image processing on the image; and displaying the image-processed image and the calculation result in synchronization with each other.

A motion state monitoring program according to an aspect of the present disclosure is a motion state monitoring program for causing a computer included in a motion state monitoring system to monitor a motion state at a target part of a subject's body, the motion state monitoring system including: an arithmetic processing unit configured to generate a calculation result representing the motion state based on a detection result of a sensor configured to detect the motion state; an image processing unit configured to perform image processing on at least one of a photographed image obtained by photographing the motion state and a drawn image obtained by drawing the motion state; and a display unit configured to display the image-processed image and the calculation result in synchronization with each other, and the motion state monitoring program being configured to cause the computer to perform: generating the calculation result representing the motion state based on the detection result; performing image processing on the image; and displaying the image-processed image and the calculation result in synchronization with each other.

According to the present disclosure, it is possible to provide a motion state monitoring system, a motion state monitoring method, and a motion state monitoring program capable of facilitating the comprehension of a motion state at a target part of a subject's body when data thereon is reviewed.

The above and other objects, features and advantages of the present disclosure will become more fully understood from the detailed description given hereinbelow and the accompanying drawings.

### BRIEF DESCRIPTION OF DRAWINGS

FIG. 1 is a structural diagram showing an example of a motion state monitoring system according to a first embodiment;

FIG. 2 is a structural diagram showing an example of the attachment of a sensor of a measuring instrument in the motion state monitoring system according to the first embodiment;

FIG. 3 is a structural diagram showing an example of the attachment of a sensor of a measuring instrument in the motion state monitoring system according to the first embodiment;

FIG. 4 is a block diagram showing an example of measuring instruments and a motion state monitoring apparatus in the motion state monitoring system according to the first embodiment;

FIG. 5 is a flowchart showing an example of a measuring method in a motion state monitoring method using the motion state monitoring system according to the first embodiment;

FIG. 6 is a flowchart showing an example of a display method in the motion state monitoring method using the motion state monitoring system according to the first embodiment;

FIG. 7 shows an example of a display on a display unit in the motion state monitoring system according to the first embodiment;

FIG. 8 shows an example of a display on the display unit in the motion state monitoring system according to the first embodiment;

FIG. 9 shows an example of a display on the display unit in the motion state monitoring system according to the first embodiment;

FIG. 10 shows an example of a display on the display unit in the motion state monitoring system according to the first embodiment;

FIG. 11 shows an example of a display on the display unit in the motion state monitoring system according to the first embodiment;

FIG. 12 shows an example of a display on the display unit in the motion state monitoring system according to the first embodiment; and

FIG. 13 is a general configurational diagram showing an example of a motion state monitoring apparatus including a computer according to an embodiment.

### DESCRIPTION OF EMBODIMENTS

The present disclosure will be described hereinafter through embodiments, but the present disclosure according to the claims is not limited to the below-shown embodiments. Further, not all of the components/structures described in the embodiments are indispensable as means for solving the problem. For clarifying the description, the following description and drawings are partially omitted and simplified as appropriate. Further, the same symbols are assigned to the same or corresponding components throughout the drawings, and redundant descriptions thereof are omitted as appropriate.

### First Embodiment

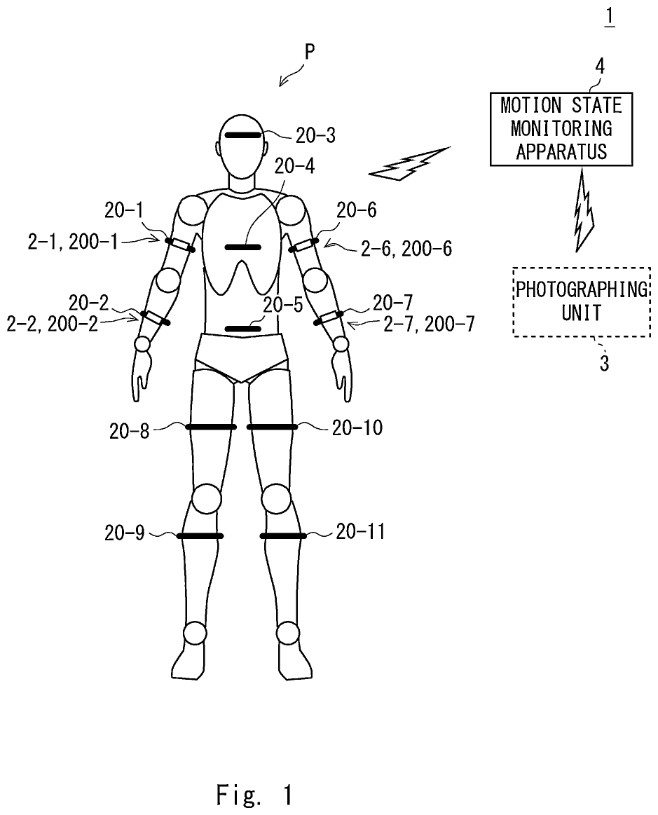

A motion state monitoring system 1 according to a first embodiment will be described hereinafter. FIG. 1 is a structural diagram showing an example of a motion state monitoring system according to the first embodiment. The motion state monitoring system 1 monitors a motion state of a part of the body of a subject P. Further, the motion state monitoring system 1 supports the motion performed by the subject P based on the monitoring result of the motion of the subject P so that the motion of the subject P becomes closer to a desired motion. Specifically, the motion state monitoring system 1 measures the motion function of the subject P, who is, for example, a rehabilitation trainee or an elderly person. Then, the motion state monitoring system 1 supports the training performed by the subject P by analyzing, evaluating, and managing the results of the measurement. The motion state monitoring system 1 displays the measurement results to be analyzed, evaluated, and managed on a display unit. The subject P performs a motion test with sensors 200 attached to predetermined parts of his/her body. For example, the motion test is a motion function test for measuring the motion state at a part of the body of the subject P when the subject P performs a designated motion, thereby measuring his/her motion function. Examples of the motion state monitoring system 1 includes a training support system.

Hereinafter, the designated motion is referred to as a monitoring target motion. The monitoring target motion is determined corresponding to the part of the body. Examples of the monitoring target motion include flexion and extension of shoulder, adduction and abduction of shoulder, lateral and medial rotations of shoulder, flexion and extension of neck, medial rotation of neck, flexion and extension of elbow, lateral and medial rotation of hip, pronation and external rotation of forearm, and thoracolumbar lateral flexion. When the target part is either left or right body part, the monitoring target motion may be separately determined for the left or right body part. A part to be monitored is referred to as a target part. As the target part, one or a plurality of parts may be associated with one monitoring target motion, and the same part may be associated with different monitoring target motions.

As shown in FIG. 1, the motion state monitoring system 1 includes measuring instruments 2 and a motion state monitoring apparatus 4. The motion state monitoring apparatus 4 may be referred to as the motion state monitoring system 1. The motion state monitoring apparatus 4 is an apparatus that monitors the motion state at the target part of the body of the subject P. Hereinafter, the motion state at the target part of the body of the subject P is referred to as the motion state of the target part. The motion state monitoring system 1 may include a photographing unit 3. Note that measuring instruments 2-1, 2-2, . . . , and the like shown in the drawing are collectively referred to as the measuring instruments 2.

The measuring instruments 2 measure the motion states of the target parts. Specifically, the measuring instrument 2 (i.e., each measuring instrument 2) a measuring apparatus that measures the moving direction and the moving amount of the target part. The measuring instrument 2 (i.e., each measuring instrument 2) includes a sensor 200 for detecting a motion state. In this embodiment, the measuring instrument 2 may include an acceleration sensor and an angular velocity sensor. The measuring instrument 2 measures the acceleration and angular velocity of the measuring instrument 2 itself. Specifically, the measuring instrument 2 may include a three-axis acceleration sensor and a three-axis angular velocity sensor. In this case, the measuring instrument 2 measures moving amounts in the three-axis directions, i.e., the XYZ-axis directions, and rotation angles around the three axes. Note that the measurement axes are not limited to three axes, and instead may be two or fewer axes. Further, the measuring instrument 2 may include a geomagnetic sensor for detecting geomagnetism and measuring a direction in which the measuring instrument 2 itself is oriented. Note that sensors 200-1, 200-2, . . . , and the like shown in the drawing are collectively referred to as the sensors 200.

Each of the measuring instruments 2 is connected to the motion state monitoring apparatus 4 so that they can communicate with each other. In this embodiment, communication between each measuring instrument 2 and the motion state monitoring apparatus 4 is short-range wireless communication such as Bluetooth (Registered Trademark), NFC (Near Field Communication) or ZigBee. However, the communication is not limited to these examples, and may be wireless communication through a network such as a wireless LAN (Local Area Network). Further, the communication may be wired communication through the Internet, a LAN, a WAN (Wide Area Network), or a network formed by a combination thereof.

The measuring instrument 2 (i.e., each measuring instrument 2) includes, in addition to the sensor 200, an attaching mechanism for the sensor 200. Further, the sensor 200 is attached to an attaching position 20 corresponding to the target part of the body of the subject P through the attaching mechanism. Note that in order to cope with the measurement of various monitoring target parts, each of a plurality of sensors 200 is associated with a respective one of a plurality of target parts of the body of the subject P and can be attached to the associated target part. In the drawing, the target parts at which the sensors 200 can be attached are indicated as attaching positions 20-1, 20-2, . . . , and 20-11. Each of the attaching positions 20-1, 20-2, . . . , and 20-11 is associated with a respective one of the sensors 200-1, 200-2, . . . , and 200-11. For example, attaching positions 20-1, 20-2, . . . , and 20-11 are referred to as the right upper arm, right forearm, head, chest (trunk), waist (pelvis), left upper arm, left forearm, right thigh, right lower leg, left thigh, and left lower leg, respectively. The associations between the attaching positions 20 and the sensors 200 are made by pairing between the sensors 200 and the motion state monitoring apparatus 4 in advance and associating identification information (ID) of the attaching positions 20 with the IDs of the sensors 200 in an application of the motion state monitoring apparatus 4. Note that the attaching positions 20-1, 20-2, . . . , and the like are collectively referred to as the attaching positions 20.

In this embodiment, the attaching positions 20 used in the motion test is selected from the attaching positions 20-1 to 20-11 according to the monitoring target motion selected by a user. Note that the user refers to a user who uses the motion state monitoring apparatus 4 and is, for example, the subject P himself/herself or a staff member who carries out the motion test. Further, the subject P or the staff member attaches the sensors 200 (in this drawing, the sensors 200-1, 200-2, 200-6 and 200-7) associated with the selected attaching positions 20 (in this drawing, attaching positions 20-1, 20-2, 20-6 and 20-7) of the body of the subject P, and then starts the motion test.

Note that although the plurality of sensors 200 associated with the plurality of attaching positions 20, respectively, are prepared in the above example, the number of prepared attaching positions 20 may be one. Further, the number of prepared sensors 200 may be one.

The sensor 200 (e.g., each sensor 200) starts measurement in response to the start of the motion test and transmits sensing information to the motion state monitoring apparatus 4. The sensing information may include acceleration information, angular velocity information, or quaternion information. Further, the sensing information may include a component in each of the measurement axis directions (X, Y and Z-axis directions). Further, the sensor 200 stops the measurement in response to the end of the motion test.

The photographing unit 3 photographs the motion state of the target part. The photographing unit 3 outputs the photographed image to the motion state monitoring apparatus 4. The photographing unit 3 includes, for example, a camera. The photographing unit 3 may take a moving image or may take a still image. Therefore, the image may be a moving image or a still image. The image obtained by photographing the motion state at the target part of the body of the subject P is referred to as a subject photographed image. The subject photographed image may be simply referred to as a photographed image.

The photographing unit 3 is connected to the motion state monitoring apparatus 4 so that they can communicate with each other. In this embodiment, the communication between the photographing unit 3 and the motion state monitoring apparatus 4 may be selected from various types of wireless communication and wired communication described above in the description of the communication between the measuring instrument 2 and the motion state monitoring apparatus 4.

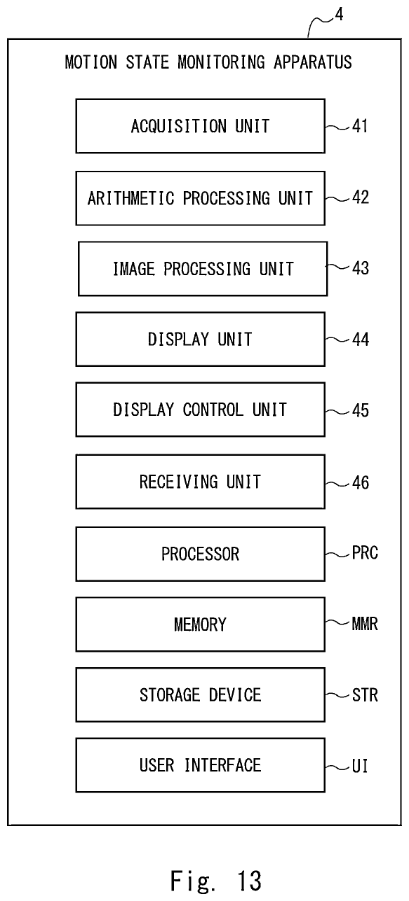

Note that the photographing unit 3 is not an indispensable component of the motion state monitoring system 1. For example, the photographing unit 3 may not be provided in the motion state monitoring system 1 as long as the motion state monitoring apparatus 4 can acquire a subject photographed image. For example, a subject photographed image(s) may be stored in a predetermined storage device (e.g., a storage device STR shown in FIG. 13), and the motion state monitoring apparatus 4 may acquire the subject photographed image from the storage device. The motion state monitoring apparatus 4 monitors the motion states of the target parts during the motion test. The motion state monitoring apparatus 4 analyzes, evaluates, and manages information about the motion states. Examples of the motion state monitoring apparatus 4 include a computer apparatus. Specifically, examples of the motion state monitoring apparatus 4 may include a personal computer, a notebook computer, a mobile phone, a smartphone, a tablet- type terminal, or other communication terminal apparatus capable of receiving/outputting data. Further, examples of the motion state monitoring apparatus 4 may include a server computer. This embodiment is described under the assumption that the motion state monitoring apparatus 4 is a tablet-type terminal.

The motion state monitoring apparatus 4 is used by the user during the motion test, and before and after the motion test. The motion state monitoring apparatus 4 receives the choice of the monitoring target motion from the user, and notifies the user of the attaching positions 20 corresponding to the target parts. Then, the motion state monitoring apparatus 4 transmits a request for starting or stopping the measurement to the sensors 200 in response to the start or end of the motion test.

Further, the motion state monitoring apparatus 4 outputs sensing-related information as the measurement result in response to the reception of the sensing information from the sensors 200. Note that the sensing-related information indicates information related to the sensing information, and may include the sensing information itself. Alternative, the sensing-related information may be information obtained by performing various conversion processes on the sensing information. Further, the sensing-related information may be information obtained by performing arithmetic processing based on the sensing information. The sensing-related information may include information about the above-described motion state. Note that the information related to the above-described motion state may include sensing-related information. That is, the information related to the above-described motion state may be information based on the sensing-related information or may include the sensing-related information itself.

The motion state monitoring apparatus 4 may be connected to an external server (not shown) through a network so that they can communicate with each other. The external server may be a computer apparatus or a cloud server on the Internet. In this case, the motion state monitoring apparatus 4 may transmit the sensing- related information or the information about the motion state of the subject P held by the motion state monitoring apparatus 4 itself to the external server.

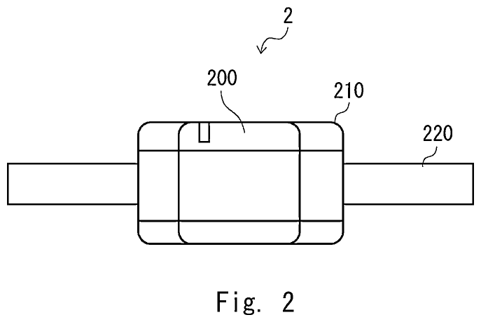

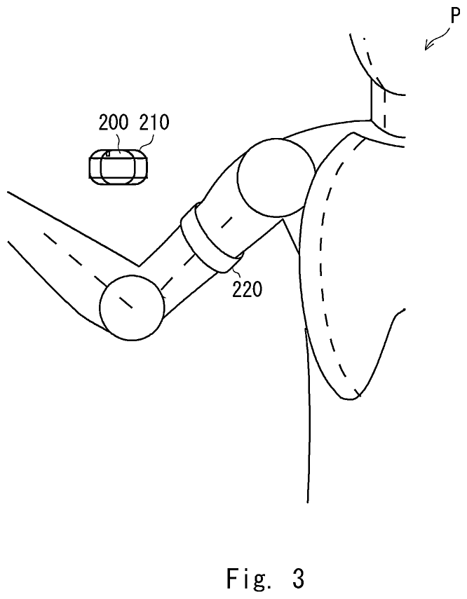

FIGS. 2 and 3 are structural diagrams showing an example of the attachment of the sensor 200 of the measuring instrument 2 in the motion state monitoring system 1 according to the first embodiment. As shown in FIG. 2, the measuring instrument 2 includes the sensor 200, an attaching pad 210, and a band 220. The band 220 is formed so that it can be wound around the target part of the subject P. The sensor 200 is incorporated, for example, into the attaching pad 210. The attaching pad 210, into which the sensor 200 is incorporated, is formed so that it can be attached to and detached from the band 220.

As shown in FIG. 3, the band 220 is wound around the right upper arm, which is one of the target parts of the subject P. The sensor 200 is attached to the band 220 through the attaching pad 210 after the completion of pairing, calibration, and the like.

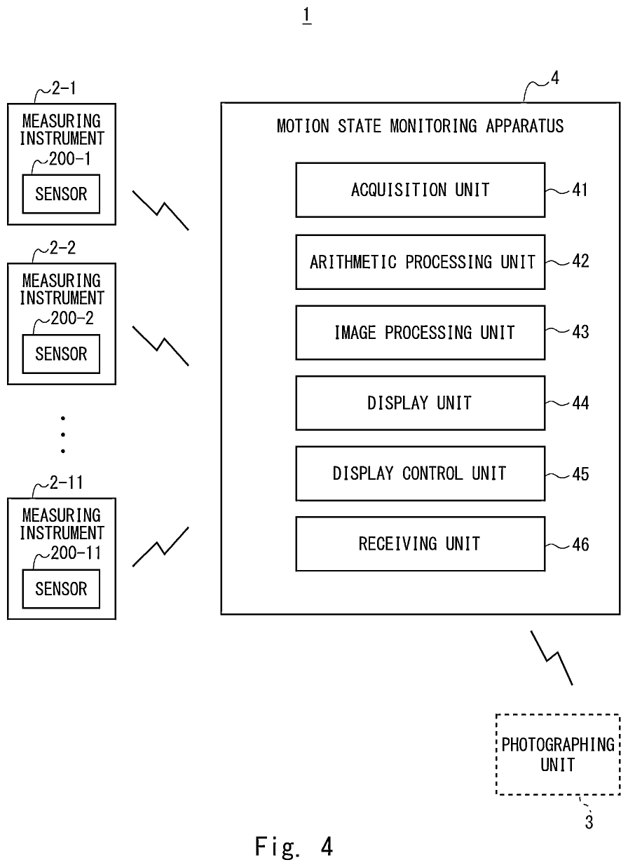

FIG. 4 is a block diagram showing an example of measuring instruments 2 and the motion state monitoring apparatus 4 in the motion state monitoring system 1 according to the first embodiment. As described above, the motion state monitoring system 1 includes the measuring instruments 2 and the motion state monitoring apparatus 4. The measuring instrument 2 (i.e., each measuring instrument 2) includes a sensor 200. In the drawing, the sensors 200 are, among the prepared sensors 200-1 to 200-11, sensors 200 associated with the attaching positions 20 selected based on the monitoring target motion. It is assumed that the sensors 200 have been paired with the motion state monitoring apparatus 4 and calibrated in advance. Note that the number of sensors 200 is not limited to one, but may be two or more.

The motion state monitoring apparatus 4 includes an acquisition unit 41, an arithmetic processing unit 42, an image processing unit 43, a display unit 44, a display control unit 45, and a receiving unit 46. The acquisition unit 41, the arithmetic processing unit 42, the image processing unit 43, the display unit 44, the display control unit 45, and the receiving unit 46 function as acquisition means, arithmetic processing means, image processing means, display means, display control means, and receiving means, respectively.

The acquisition unit 41 acquires detection results of the sensors 200. Specifically, the acquisition unit 41 acquires sensing information from the sensors 200 attached to the target parts. Further, the acquisition unit 41 acquires a subject photographed image. For example, the acquisition unit 41 acquires the subject photographed image from the photographing unit 3. Note that the acquisition unit 41 may acquire the subject photographed image from a storage device in which the subject photographed image is stored in advance.

Further, the acquisition unit 41 may acquire an image obtained by drawing the motion state of the target part. The image obtained by drawing the motion state of the target part is referred to as a subject drawn image. Note that the subject drawn image may be simply referred to as a drawn image. The image processing unit 43 may generate the subject drawn image from the subject photographed image. The acquisition unit 41 acquires the subject drawn image from the image processing unit 43. Note that the acquisition unit 41 may acquire the subject drawn image from a storage device in which the subject drawn image is stored in advance. Further, the acquisition unit 41 acquires information input from the user by the receiving unit 46.

Based on the detection results of the sensors 200, the arithmetic processing unit 42 generates a calculation result representing the motion state at the target part of the body of the subject P. The detection results of the sensors 200 also include, for example, the angle of a joint of the body of the subject P detected in the monitoring target motion and the angle of a joint in an arbitrary coordinate system measured based on the detection result of one of the sensors. Hereinafter, the generation of a calculation result representing the motion state of the monitoring target motion is also referred to as the measurement of the monitoring target motion.

For example, the arithmetic processing unit 42 performs arithmetic processing based on the detection results of, among the sensors 200-1 to 200-11, the sensor 200-1 attached to the right upper arm (the attaching position 20-1) and the sensor 200-2 attached to the right forearm (the attaching position 20-2) of the subject P. In this way, the arithmetic processing unit 42 generates a calculation result representing the motion state of the flexion and extension of the right elbow of the subject P.

Alternatively, the arithmetic processing unit 42 performs arithmetic processing based on the detection results of, among the sensors 200-1 to 200-11, of the sensor 200-5 attached to the waist (the attaching position 20-5) and the sensor 200-8 attached to the right thigh (the attaching position 20-8) of the subject P. In this way, the arithmetic processing unit 42 generates a calculation result representing the motion state of the lateral flexion of the waist on the right side of the subject P.

Note that the arithmetic processing unit 42 may perform arithmetic processing by using a trained model generated through machine learning using past detection results of the sensors 200. By performing such arithmetic processing using a trained model, the arithmetic processing unit 42 can more accurately calculate whether or not the motion state of the monitoring target motion of the subject P is satisfactory.

The arithmetic processing unit 42 may perform a process for visualizing the calculation result in the form of a graph, a table, or the like. In this way, the user can easily comprehend the motion state of the target part. Therefore, for example, the calculation result or the like can be used for assisting the subject P.

The image processing unit 43 processes the subject photographed image. Further, the image processing unit 43 processes the subject drawn image. The image processing unit 43 processes at least one of the subject photographed image and the subject drawn image.

The image processing unit 43 may generate a subject drawn image of a 3D model obtained by drawing the motion state of the subject P. The image processing unit 43 may generate a subject drawn image of a 3D model obtained by drawing the motion state shown in the subject photographed image. Further, the image processing unit 43 may also generate a subject drawn image of a 3D model obtained by drawing the motion state, which is used as a reference, in advance. The motion state used as the reference includes an ideal motion state of the target part of the subject P. Note that the image processing unit 43 may generate a subject drawn image of an avatar instead of the 3D model. The avatar may include an animated image. As described above, the subject drawn image is not necessarily limited to the 3D model and may include a drawn image of an avatar.

The image processing unit 43 may generate a locus of the target part in the subject drawn image of the 3D model. For example, the image processing unit 43 may generate a regulation(s) for the target part in the subject drawn image of the 3D model obtained by drawing the motion state shown in the subject photographed image. Further, the image processing unit 43 may generate a locus of the target part in the subject photographed image. Further, the image processing unit 43 may also generate a locus of the target part in the subject drawn image of the 3D model obtained by drawing the motion state which is generated in advance and used as the reference.

The image processing unit 43 may generate a Lissajous figure in which a plurality of loci are combined with each other. Specifically, for example, the image processing unit 43 generates, as a first locus, a locus of the target part in the subject drawn image of the 3D model obtained by drawing the motion state shown in the subject photographed image. Further, the image processing unit 43 generates, as a second image, a locus of the target part in the subject drawn image of the 3D model obtained by drawing the motion state which is generated in advance and used as the reference. Then, the image processing unit 43 generates a Lissajous figure in which the first and second loci are combined with each other.

The image processing unit 43 may generate a figure indicating a movable area of the target part in the subject drawn image of the 3D model.

The display unit 44 displays the calculation result of the motion state of the target part generated by the arithmetic processing unit 42. Further, the display unit 44 displays at least one of the subject photographed image and the subject drawn image, which have been image-processed by the image processing unit 43. For example, the display unit 44 displays the subject drawn image of the 3D model generated by the image processing unit 43. The display unit 44 may display the subject drawn image of the 3D model in an enlarged or reduced manner, and may display the subject photographed image in an enlarged or reduced manner.

The display unit 44 displays the subject drawn image including the locus of the target part generated by the image processing unit 43. The display unit 44 may display the subject photographed image including the locus of the target part generated by the image processing unit 43. The display unit 44 displays at least one of the subject photographed image and the subject drawn image, and the calculation result in synchronization with each other.

The display unit 44 may display the Lissajous figure generated by the image processing unit 43. Further, the display unit 44 may display the generated figure indicating the movable area. The display unit 44 may display the Lissajous figure, the figure indicating the movable area, and the calculation result in synchronization with each other.

The display unit 44 may display at least one of vital data and a walking speed. Further, the display unit 44 may display a plurality of subject photographed images and a plurality of subject drawn images obtained at different times. The display unit 44 displays the calculation result, and the subject photographed image and the subject drawn image and the like under the control of the display control unit 45.

The display control unit 45 controls the display of the display unit 44. Specifically, the display control unit 45 controls the display unit 44 so as to display the calculation result generated by the arithmetic processing unit 42, the subject photographed image and the subject drawn image and the like image-processed by the image processing unit 43 thereon.

The receiving unit 46 receives information input by the user.

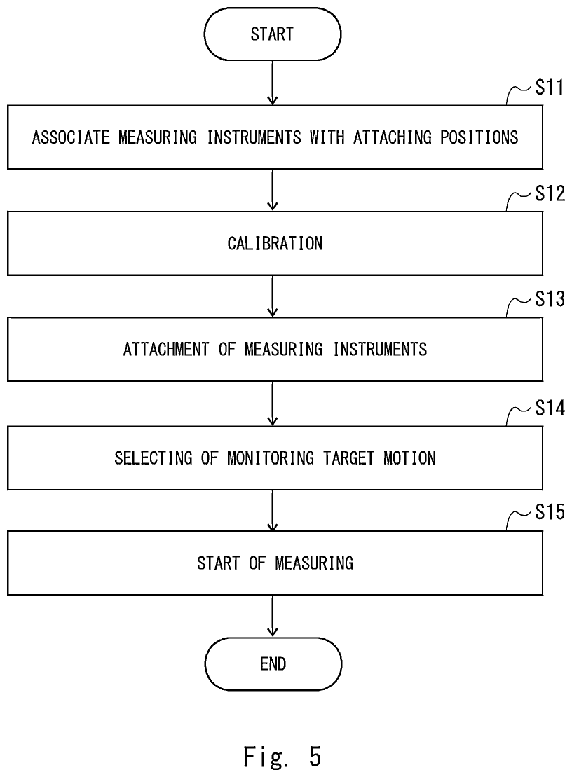

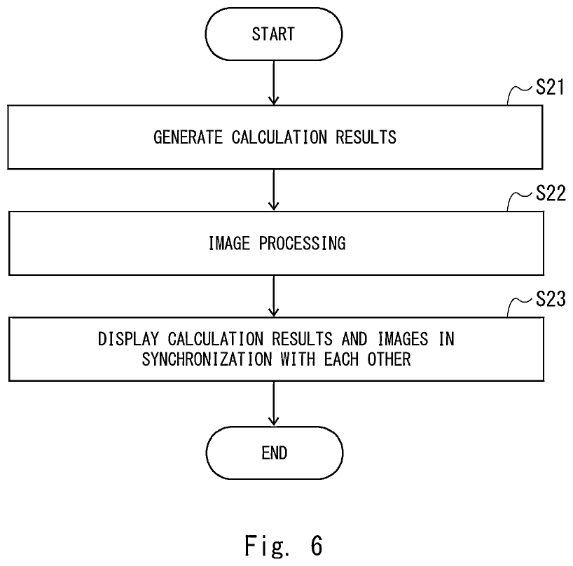

Next, a motion state monitoring method using the motion state monitoring system 1 will be described. FIG. 5 is a flowchart showing an example of a measurement method in the motion state monitoring method using the motion state monitoring system 1 according to the first embodiment. FIG. 6 is a flowchart showing an example of a display method in the motion state monitoring method using the motion state monitoring system 1 according to the first embodiment.

As shown in a step S11 in FIG. 5, in the motion state monitoring system 1, measuring instruments 2 and attaching positions 20 are associated with each other. In this way, it is possible to perform a pairing process between the motion state monitoring apparatus 4 and the measuring instruments 2.

Next, as shown in a step S12, calibration of the sensors 200 in the measuring instruments 2 is performed. The calibration is, for example, a process for measuring an output value (error component) of the sensor 200 used for the measurement of the monitoring target motion in a stationary state and subtracting the measured error component from an actual measured value. Note that it is assumed that the output value of the sensor 200 is stabilized after a predetermined period (about 20 seconds) has elapsed after the sensor 200 is kept stationary. In this case, an output value of the sensor 200 that is output after the predetermined period has elapsed after the sensor 200 is kept stationary is preferably used as an error component in the calibration. Therefore, in this embodiment, an output value of the sensor 200 that is output after a predetermined period has elapsed after the user provides an instruction to start the calibration after the sensor 200 is kept stationary is used as an error component. Further, the expression “during calibration” means a processing period until the error component is determined, and the expression “the completion of calibration” means that the output value (error component) of the sensor in the stationary state is determined.

During the calibration, for example, “Calibration is in progress” is displayed on the display unit 44. When the calibration is completed, for example, “Calibration has been completed” is displayed on the display unit 44. Note that the display of the information indicating that the calibration is in progress or that the calibration has been completed is not limited to the display on the display unit 44, and may be reported by other notification methods such as by voice or sound.

Next, as shown in a step S13, the measuring instruments 2 are attached to the subject P. In this embodiment, among the sensors 200-1 to 200-11, the sensors 200-1, 200-2, 200-6, and 200-7 are attached to the right upper arm (attaching position 20-1), the right forearm (attaching position 20-2), the left upper arm (attaching position 20-6), and the left forearm (attaching position 20-7), respectively, of the subject P. Each of the sensors 200 may be attached to the respective attaching position through the attaching pad 210 and the band 220.

Next, as shown in a step S14, among a plurality of monitoring target motions, those can be measured by using the sensors 200 attached to the subject P are measured.

Next, as shown in a step S15, the motion state monitoring apparatus 4 starts measuring the motion states of the target parts.

Next, a display method will be described. The display by the display unit 44 according to this embodiment may be performed during the measurement in the motion test, or may be performed before the measurement starts and after the measurement ends in the motion test.

As shown in a step S21 of FIG. 6, the arithmetic processing unit 42 generates a calculation result representing the motion state of the target part based on the detection result of the sensor that detects the motion state. The generated calculation result representing the motion state includes, for example, the measurement result of the monitoring target motion.

Next, as shown in a step S22, the image processing unit 43 performs image processing on at least one of the subject photographed image obtained by photographing the motion state of the target part and the subject drawn image obtained by drawing the motion state.

Next, as shown in a step S23, the display unit 44 displays the image-processed image and the calculation result in synchronization with each other.

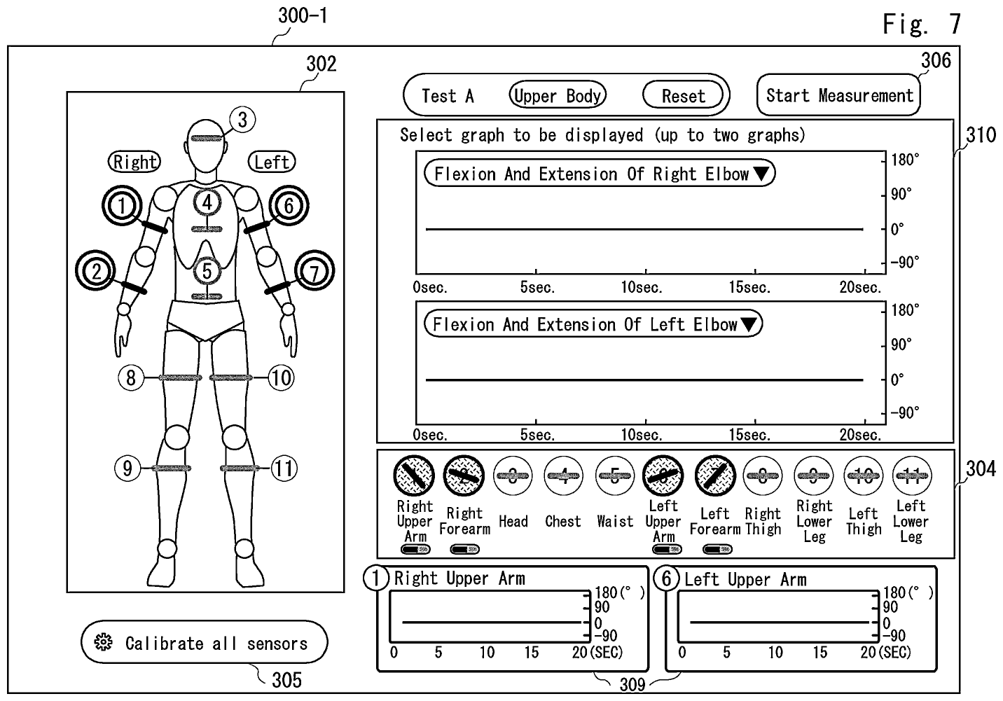

FIGS. 7 to 12 show examples of the displays on the display unit 44 in the motion state monitoring system 1 according to the first embodiment. FIG. 7 shows a display image 300-1 before the measurement starts in the motion test. As shown in FIG. 7, the display image 300-1 includes display areas 302, 304, 305, 306, 309 and 310.

In the display area 302, a plurality of icon images representing respective attaching positions 20 that are candidates for positions where the sensors 200 are attached. In the display area 302, the attaching positions 20 corresponding to the selected measurement motion (positions indicated by “1”, “2”, “6” and “7” in the drawing) may be highlighted. In this way, the user can easily visually recognize the attaching positions 20, so that he/she can smoothly carry out the motion test.

In the display area 304, the rotation angle of each of the sensors 200-1, 200-2, . . . , and 200-11 associated with the respective attaching positions 20-1, 20-2, . . . , and 20-11 is displayed in a two-dimensional manner. Note that the displayed rotation angles dynamically change in response to the motions of the sensors 200 in conjunction with the motion of the subject P. Therefore, the user can specify a sensor(s) 200 that is turned off or a sensor(s) 200 that is not properly operating through the display area 304 before the start of the measurement.

In the display area 305, an input operation button for, when a plurality of sensors 200 are used for the motion test, calibrating the plurality of sensors 200 at once is displayed. In this way, the user can easily request the calibration of each of the plurality of sensors 200 through the display area 305.

In the display area 306, an input operation button for starting the motion test, i.e., starting the measurement by the sensors 200, is displayed. In this way, the user can easily request to start the measurement by the sensors 200 through the display area 306.

In the display area 309, sensing-related information of each of used sensors 200 is displayed. Any sensing-related information has not been displayed yet because the measurement has not started yet. In the display area 310, motion state indices of the target parts are displayed for each performed monitoring target motion. Any motion state index of any of the target parts has not been displayed yet because the measurement has not started yet.

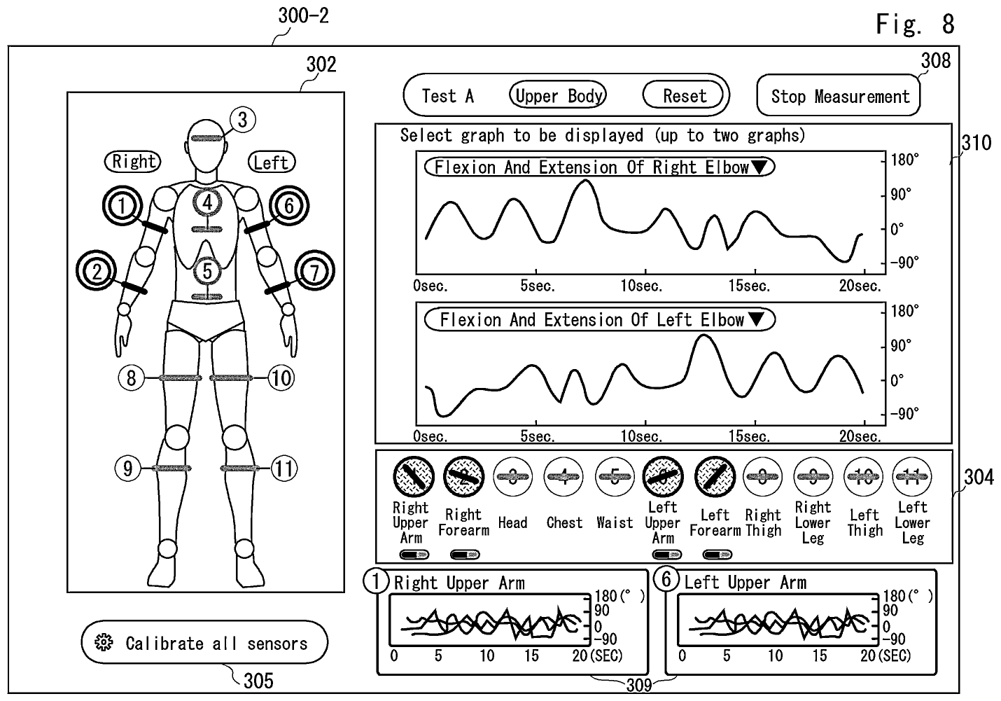

FIG. 8 shows a display image 300-2 at the end of the measurement in the motion test. Similarly to the display image 300-1, the display image 300-2 includes display areas 302, 304, 305, 306, 309 and 310. The display areas 302 and 304 of the display image 300-2 are similar to the display areas 302 and 304 of the display image 300-1 shown in FIG. 6.

In the display area 308, an input operation button for terminating the motion test, i.e., stopping measurement by the sensors 200, is displayed. In this way, the user can easily request to stop the measurement by the sensors 200 through the display area 308.

In the display area 309, sensing-related information for each of the used sensors 200 is displayed. The rotation angles around the Xs, Ys and Zs-axes, which have been determined based on the outputs of some of the sensors among the used sensors 200-1, 200-2, 200-6, and 200-7, i.e., the outputs of the sensors 200-1 and 200-6, are displayed in a chronological order.

In the display area 310, motion state indices of the target parts are displayed for each performed monitoring target motion. The motion state index is an index indicating the motion state of the target part when the monitoring target motion is performed. The arithmetic processing unit 42 calculates the motion state index of the target part based on the sensing-related information of the sensor 200. For example, when the monitoring target motion is “flexion and extension of right elbow”, the sensing-related information of each of the sensors 200-1 and 200-2 at the attaching positions 20-1 and 20-2, respectively, is used. In this case, the arithmetic processing unit 42 may calculate the motion state index based on the difference between the sensing-related information of the sensor 200-1 and that of the sensor 200-2. In the display area 310, the motion state indices of some of the plurality of performed monitoring target motions are displayed in a chronological order.

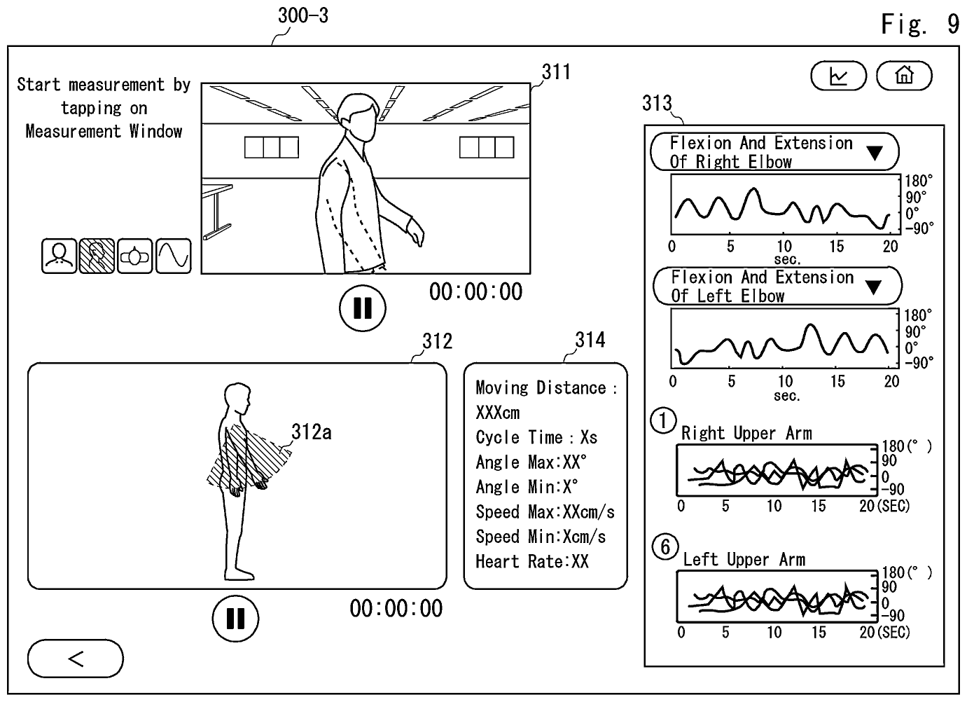

FIG. 9 shows a display image 300-3. The display image 300-3 includes display areas 311 to 314. A subject photographed image is displayed in the display area 311. The subject photographed image may be a moving image. A subject drawn image is displayed in the display area 312. The subject drawn image displayed in the display area 312 includes a subject drawn image of a 3D model generated by the image processing unit 43. The subject drawn image of the 3D model may be generated by drawing the motion state shown in the subject photographed image. In the display area 313, calculation results representing motion states generated by the arithmetic processing unit 42 are displayed based on the detection results of the sensors 200. The calculation results include, for example, changes in the rotation angle over time shown in the form of a graph.

As described above, the display unit 44 may display the subject photographed image and the subject drawn image. The display unit 44 may display a drawn image of a 3D model as the subject drawn image. The display unit 44 may display the subject photographed image and the subject drawn image in synchronization with each other. That is, the display unit 44 may display the subject photographed image and the subject drawn image in such a manner that the time at which the subject photographed image was photographed and the time at which the subject photographed image used to generate the subject drawn image was photographed are synchronized with each other. In this way, the user can observe the motion states of the target parts by associating corresponding positions in a 3D model with the target parts. Therefore, it is possible to facilitate the comprehension of the motion states.

The display unit 44 may display the subject photographed image displayed in the display area 311 in an enlarged or reduced manner, or may display the subject drawn image displayed in the display area 312 in an enlarged or reduced manner. The display unit 44 may display the subject drawn image of the 3D model in an enlarged or reduced manner. In this way, it is possible to facilitate the comprehension of the motion states even further.

In the display area 312, a FIG. 312a indicating the movable area of the target part in the subject drawn image of the 3D model generated by the image processing unit 43 is displayed. Further, in the display area 314, vital data and a walking speed are displayed. In this way, the display unit 44 may display the generated FIG. 312a indicating the movable area, and may further display at least one of vital data and a walking speed. By the above-described display method, the user or the like can comprehend the motion states in a more detailed manner.

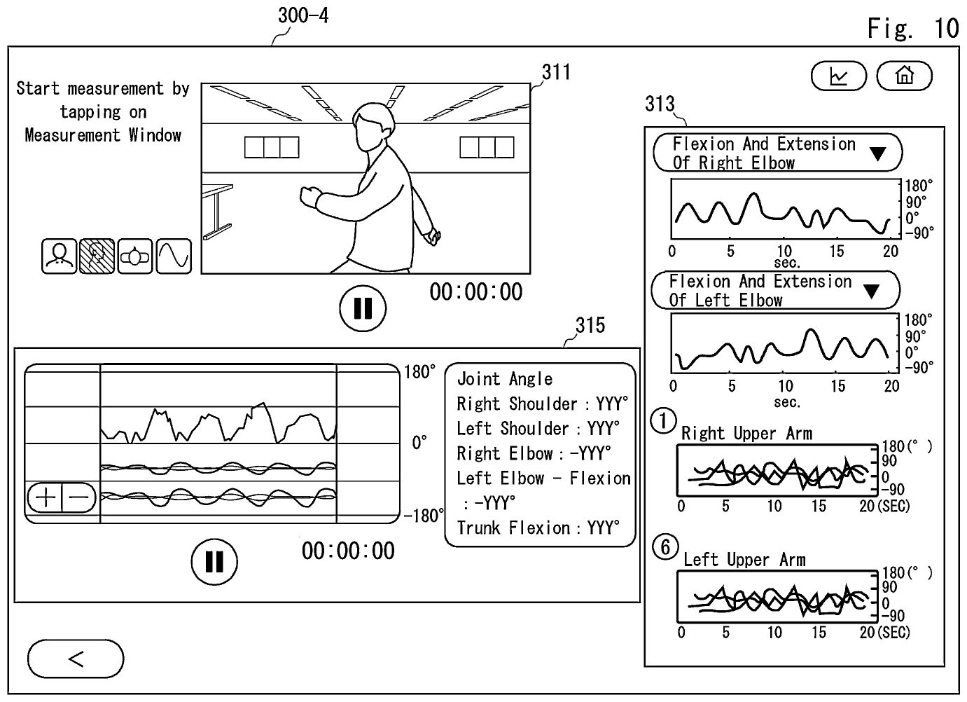

FIG. 10 shows a display image 300-4. The display image 300-4 includes display areas 311, 313 and 315. Similar to the display area 313, in the display area 315, calculation results representing motion states generated by the arithmetic processing unit 42 are displayed. In the display area 315, a plurality of calculation results are combined and displayed in an enlarged manner.

As described above, the display unit 44 may display the subject photographed image and the calculation results. Note that the display unit 44 may display the subject drawn image and the calculation results instead of or in addition to the subject photographed image. The display unit 44 may display at least one of the subject photographed image and the subject drawn image, and the calculation results in synchronization with each other. That is, the display unit 44 may display the subject photographed image while synchronizing the time at which the subject photographed image was photographed and the time at which the measurement was performed by the sensor 200 with each other. In this way, the user can observe the motion states of the target parts by associating corresponding positions in a 3D model with the target parts, and hence can easily comprehend the motion states.

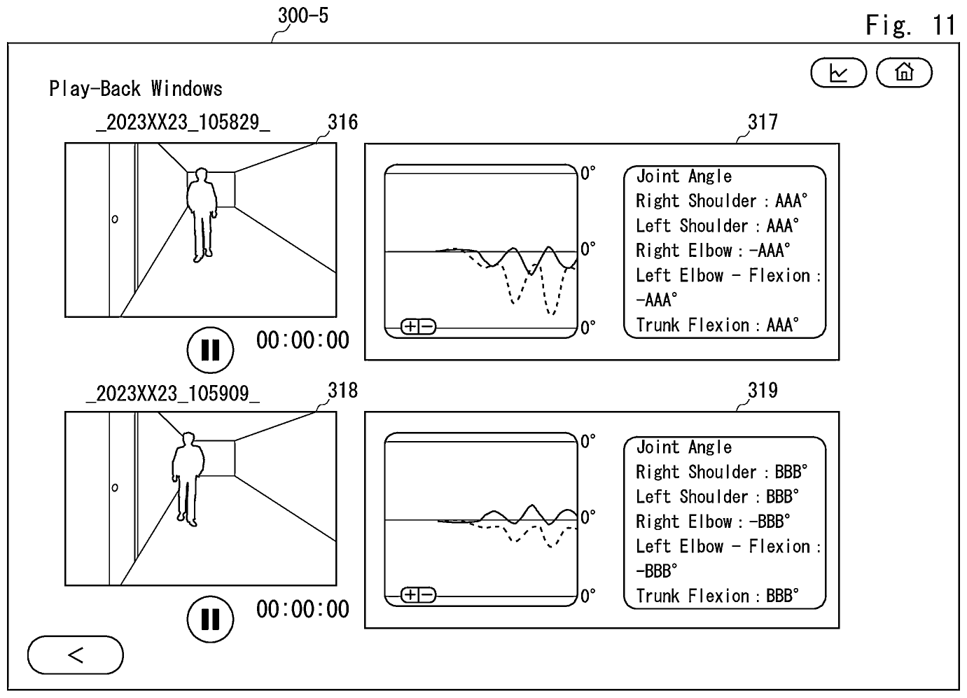

FIG. 11 shows a display image 300-5. The display image 300-5 includes display areas 316 to 319. In the display area 316, a subject photographed image taken at a predetermined photographing time is displayed. For example, a subject photographed image including a motion state of a target part when the motion state is not satisfactory is displayed. In the display area 317, calculation results are displayed in synchronization with the subject photographed image displayed in the display area 316. In the display area 318, a subject photographed image taken at a photographing time different from the predetermined photographing time is displayed. For example, a subject photographed image after the motion state has been satisfactorily recovered is displayed. In the display area 319, calculation results are displayed in synchronization with the subject photographed image displayed in the display area 318.

As described above, the display unit 44 may display a plurality of subject photographed image taken at different times. Note that the display unit 44 may display subject drawn images obtained by drawing a plurality of subject photographed images taken at different times, or may display subject photographed images and subject drawn images obtained at different times.

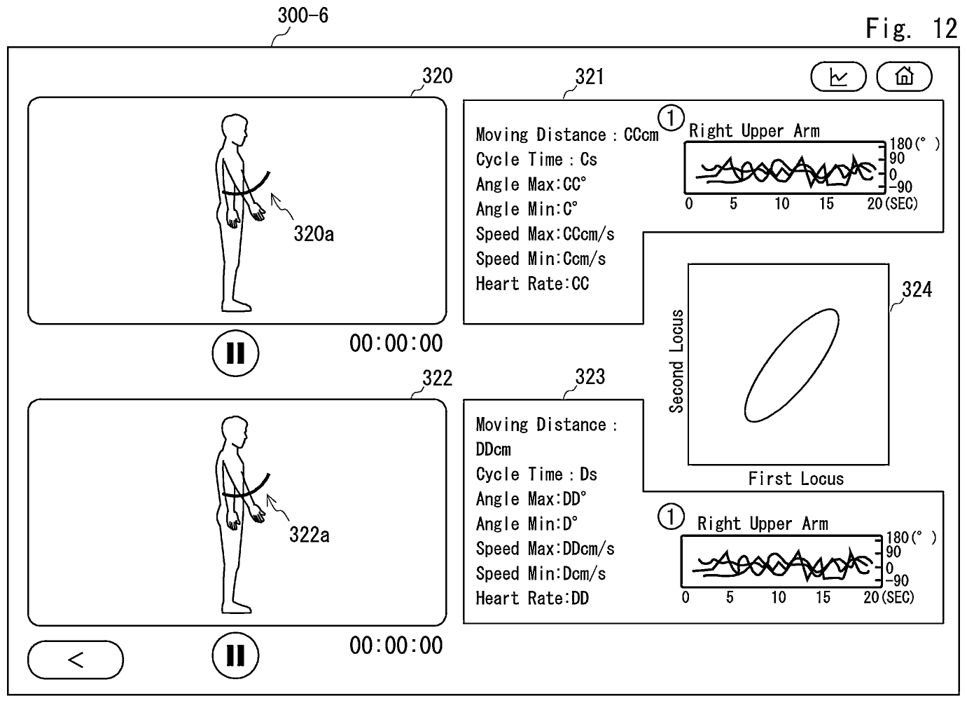

FIG. 12 shows a display image 300-6. The display image 300-6 includes display areas 320 to 324. In the display area 320, a subject drawn image of a 3D model generated by the image processing unit 43 is displayed. The subject drawn image displayed in the display area 320 includes a locus 320a of the target part. The subject drawn image displayed in the display area 320 may be a subject drawn image of a 3D model obtained by drawing the motion state shown in the photographed image. In the display area 321, calculation results are displayed in synchronization with the subject drawn image displayed in the display area 320.

In the display area 322, another subject drawn image of a 3D model is displayed. The subject drawn image displayed in the display area 322 includes a locus 322a of the target part. The subject drawn image displayed in the display area 322 may be a subject drawn image of a 3D model obtained by drawing a motion state which is generated in advance and used as a reference by the image processing unit 43. In the display area 323, calculation results are displayed in synchronization with the subject drawn image displayed in the display area 320.

As described above, the display unit 44 may display a subject drawn image including loci 320a and 322a of the target part in the subject drawn image of the 3D model. In this case, the display unit 44 may display, as the subject drawn image including the loci 320a and 322a, a subject drawn image obtained by drawing a motion state shown in a photographed image, or may display a subject drawn image of a 3D model obtained by drawing a motion state which is generated in advance and used as a reference. Note that the display unit 44 may display a subject photographed image including a locus of a target part shown in a photographed image, generated by the image processing unit 43.

In the display area 324, a Lissajous figure generated by the image processing unit 43 is displayed. The Lissajous figure is generated by combining first and second loci. The first locus is, for example, the locus 320a of the target part as displayed in the display area 320. The second locus is, for example, the locus 322a of the target part as displayed in the display area 322.

As described above, the display unit 44 may display a Lissajous figure in which the first and second loci generated by the image processing unit 43 are combined. By displaying such a Lissajous figure, it is possible to display a difference between a motion state which is generated in advance and used as a reference and a motion state at the time of the photographing, and thereby to facilitate the comprehension of the motion state.

Next, effects of this embodiment will be described. The motion state monitoring system 1 according to this embodiment displays at least one of a subject photographed image and a subject drawn image, and calculation results in synchronization with each other. Therefore, it is possible to facilitate the comprehension of the motion state of the target part.

Since the display unit 44 displays a subject drawn image of a 3D model, the motion state can be easily visually recognized. For example, the display unit 44 can display a subject drawn image in an enlarged or reduced manner. Therefore, it is possible to focus on each of the target parts, and thereby to facilitate the comprehension of the motion state. Further, the display unit 44 displays a figure indicating the movable area of the target part, vital data, a walking speed, and the like, so that it is possible to comprehend the motion state in a more detailed manner.

The display unit 44 displays a locus (loci) of the target part in the motion state and a Lissajous figure thereof. In this way, it is possible to comprehend the motion state of the target part in a more detailed manner.

Although the present disclosure has been described as a hardware configuration in the above embodiments, the present disclosure is not limited to this. According to the present disclosure, each of the processing related to the motion state monitoring method can be implemented by causing the processor to execute a computer program, for example, a motion state monitoring program.

In the embodiments described above, the computer is composed of a computer system including a personal computer, a word processor, etc. However, the computer is not limited to this and may be constituted by a LAN server, a host of computer (personal computer) communication, a computer system connected to the Internet, or the like. The functions may be distributed to devices on the network and a whole network may serve as a computer.

FIG. 13 is a general structural diagram showing an example of a motion state monitoring apparatus 4 including a computer according to an embodiment. As shown in FIG. 13, the motion state monitoring apparatus 4 may further include a processor PRC, a memory MMR, a storage device STR, and a user interface UI. In the storage device STR, a process performed by each of the components of the motion state monitoring apparatus 4 are stored in the form of a computer program(s). Further, the processor PRC loads the program from the storage device STR onto the memory MMR, and executes the loaded program. In this way, the processor PRC implements the function of each of the components in the motion state monitoring apparatus 4. The user interface UI may include an input device such as a keyboard, a mouse, and a photographing apparatus, and an output device such as a display, a printer, and a speaker.

Each of the components included in the motion state monitoring apparatus 4 may be implemented by dedicated hardware. Further, some or all of the components may be implemented by, for example, a general purpose or dedicated circuit (Circuitry), a processor PRC, or a combination thereof. These components may be formed by a single chip or a plurality of chips connected to each other through a bus. Some or all of the components may be implemented by any combination of the above-described circuits and programs. Further, as the processor PRC, a CPU (Central Processing Unit), a GPU (Graphics Processing Unit), an FPGA (Field-programmable Gate Array), a quantum processor (quantum computer control chip) or the like can be used.

Further, when some or all of the components of the motion state monitoring apparatus 4 are implemented by a plurality of information processing apparatuses, circuits, or the like, the plurality of information processing apparatuses, circuits, or the like may be centralized in one place or distributed over a plurality of places. For example, the information processing apparatuses, circuits, or the like may be implemented by a client-server system, a cloud computing system, or the like in a form in which the apparatuses or the like are connected to each other through a communication network NW. Further, the function of the motion state monitoring apparatus 4 may be provided in a Saas (Software as a Service) form.

The order of executions of processes in the apparatus and method shown in the claims, the specification, and the drawings may be implemented in any order unless it is specifically indicated as “before”, “prior to”, or the like, and unless an output of the preceding process is used in the subsequent process. Even when the flow of operations in the claims, the specification, and the drawings is explained by using a term such as “Firstly”, “Then”, or the like for the sake of convenience, it does not mean that it is necessary to implement the processes in this order.

The program can be stored and provided to a computer using any type of non- transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A motion state monitoring system configured to monitor a motion state at a target part of a subject's body, comprising:
an arithmetic processing unit configured to generate a calculation result representing the motion state based on a detection result of a sensor configured to detect the motion state;
an image processing unit configured to perform image processing on at least one of a photographed image obtained by photographing the motion state and a drawn image obtained by drawing the motion state; and
a display unit configured to display the image-processed image and the calculation result in synchronization with each other.

2. The motion state monitoring system according to claim 1, wherein
the image processing unit generates the drawn image of a 3D model obtained by drawing the motion state or the drawn image of an avatar obtained by drawing the motion state, and
the display unit displays the generated drawn image of the 3D model or the avatar.

3. The motion state monitoring system according to claim 2, wherein
the image processing unit generates a locus of the target part in the drawn image of the 3D model or the avatar, and
the display unit displays the drawn image including the generated locus.

4. The motion state monitoring system according to claim 2, wherein
the image processing unit generates a locus of the target part in the photographed image, and
the display unit displays the photographed image including the generated locus.

5. The motion state monitoring system according to claim 2, wherein the image processing unit generates a locus of the target part in the drawn image of the 3D model or the avatar, obtained by drawing the motion state shown in the photographed image, and
the display unit displays the drawn image including the generated locus.

6. The motion state monitoring system according to claim 2, wherein
the image processing unit generates a locus of the target part in the drawn image of the 3D model or the avatar, obtained by drawing the motion state, the motion state being generated in advance and used as a reference, and
the display unit displays the drawn image including the generated locus.

7. The motion state monitoring system according to claim 2, wherein
the image processing unit:
generates a first locus of the target part in the drawn image of the 3D model or the avatar, obtained by drawing the motion state shown in the photographed image;
generates a second locus of the target part in the drawn image of the 3D model or the avatar, obtained by drawing the motion state, the motion state being generated in advance as the reference; and
generates a Lissajous figure in which the first and second loci are combined with each other, and
the display unit displays the generated Lissajous figure.

8. The motion state monitoring system according to claim 2, wherein the display unit displays the drawn image of the 3D model or the avatar in an enlarged or reduced manner.

9. The motion state monitoring system according to claim 2, wherein
the image processing unit generates a figure indicating a movable area of the target part in the drawn image of the 3D model or the avatar, and
the display unit displays the generated figure indicating the movable area.

10. The motion state monitoring system according to claim 1, wherein the display unit further displays at least one of vital data and a walking speed.

11. The motion state monitoring system according to claim 1, wherein the display unit displays a plurality of images obtained at different times.

12. A motion state monitoring method for monitoring a motion state at a target part of a subject's body by using a motion state monitoring system, the motion state monitoring system comprising:
an arithmetic processing unit configured to generate a calculation result representing the motion state based on a detection result of a sensor configured to detect the motion state;
an image processing unit configured to perform image processing on at least one of a photographed image obtained by photographing the motion state and a drawn image obtained by drawing the motion state; and
a display unit configured to display the image-processed image and the calculation result in synchronization with each other, and
the motion state monitoring method comprising:
generating the calculation result representing the motion state based on the detection result;
performing image processing on the image; and
displaying the image-processed image and the calculation result in synchronization with each other.

13. A non-transitory computer readable medium storing a motion state monitoring program for causing a computer included in a motion state monitoring system to monitor a motion state at a target part of a subject's body,
the motion state monitoring system comprising:
an arithmetic processing unit configured to generate a calculation result representing the motion state based on a detection result of a sensor configured to detect the motion state;
an image processing unit configured to perform image processing on at least one of a photographed image obtained by photographing the motion state and a drawn image obtained by drawing the motion state; and
a display unit configured to display the image-processed image and the calculation result in synchronization with each other, and
the motion state monitoring program being configured to cause the computer to perform:
generating the calculation result representing the motion state based on the detection result;
performing image processing on the image; and
displaying the image-processed image and the calculation result in synchronization with each other.
